(19) United States
(12) Patent Application Publication
(10) Pub. No.: US 2024/0009536 A1
(43) Pub. Date: Jan. 11, 2024

(54) SPORTS TRAINING SYSTEM FOR CORRECT PITCHING MOTIONS AND METHOD FOR USE THEREOF

(71) Applicant: NEW TURF TECHNOLOGIES, INC., Los Angeles, CA (US)

(72) Inventors: Joseph Ferrara, Thousand Oaks, CA (US); Michael Kahn, Los Angeles, CA (US)

(21) Appl. No.: 18/371,637

(22) Filed: Sep. 22, 2023

Related U.S. Application Data
(63) Continuation-in-part of application No. 17/880,301, filed on Aug. 3, 2022.
Application No. 17/880,301 is a division of application No. 16/153,620, filed on Oct. 5, 2018, now Pat. No. 11,446,559.
(60) Provisional application No. 62/568,741, filed on Oct. 5, 2017.
(60) Provisional application No. 63/409,018, filed on Sep. 22, 2022.

(51) Int. Cl. A63B 69/00; A63B 21/00; G09B 19/00; A63B 60/46; A63B 69/36; G16H 20/30; G06V 40/20

(52) U.S. Cl. CPC A63B 69/0002; A63B 21/4037; G09B 19/0038; A63B 60/46; A63B 69/3661; G16H 20/30; A63B 69/3667; G06V 40/23; A63B 2069/0008; A63B 2225/50; A63B 2220/833; A63B 2220/56; A63B 2102/18

(57) ABSTRACT

Described is a sports training system that can be used to assist pitchers in identifying foot placement and mechanics during a pitching motion. The sports training system includes a mat and plurality of foot pads. The mat is designed to have at least a pitching plate and a plurality of grid zones, while the foot pads are used for placement in one or more of the plurality of grid zones to assist the pitcher with foot placement.

### CROSS-REFERENCE TO RELATED APPLICATIONS

This is a Continuation-in-Part application of U.S. Ser. No. 17/880,301, filed on Aug. 3, 2022, which is a divisional application of U.S. Ser. No. 16/153,620, filed on Oct. 5, 2018, now U.S. Pat. No. 11,446,559, which was a non-provisional application of U.S. Provisional Application No. 62/568,741, filed on Oct. 5, 2017.

This is ALSO a non-provisional application of U.S. Provisional Application No. 63/409,018, filed on Sep. 22, 2022, the entirety of which is incorporated herein by reference.

### BACKGROUND OF THE INVENTION

### (1) Field of Invention

The present invention relates to a sports training system and, more specifically, to a sports training mat and foot pads that can be used to assist pitchers in identifying foot placement and mechanics during a pitching motion.

### (2) Description of Related Art

Pitchers are often instructed by pitching coaches with verbal cues and instructions. However, such instructions may be difficult for the pitcher to understand and/or visualize. Thus, a continuing need exists for a new and improved sports training system to assist pitchers in learning and making correct and repetitive pitching motions.

### SUMMARY OF INVENTION

The present disclosure provides a sports training system that can be used to assist pitchers in identifying foot placement and mechanics during a pitching motion. The sports training system includes a mat and plurality of foot pads. The mat is designed to have at least a pitching plate and a plurality of grid zones, while the foot pads are used for placement in one or more of the plurality of grid zones.

In one aspect, the plurality of grid zones includes a drive-lane grid, the drive-lane grid having a series of parallel markings traveling away from the pitching plate.

In another aspect, the plurality of grid zones includes a stabilizing foot grid adjacent to the pitching plate such that the stabilizing foot grid is sandwiched between the drive-lane grid and the pitching plate.

In yet another aspect, the plurality of grid zones includes one or more sweep grids adjacent to the drive-lane grid.

In another aspect, the mat further comprises one or more sweeping arrows formed on the mat that are leading away from the pitching plate and forward through the one or more sweep grids.

Further and as can be appreciated by one in the art, the present invention also comprises a method for using the invention described herein. For example, in one aspect, the present disclosure provides a method for using a sports training mat system during pitching lessons, comprising acts of positioning one or more foot pads upon a mat, the mat having a pitching plate and a plurality of grid zones; positioning one or more feet upon the one or more foot pads; and performing a pitching motion while moving feet between the one or more foot pads.

In another aspect, the plurality of grid zones includes at least a drive-lane grid having a series of parallel markings traveling away from the pitching plate and a stabilizing foot grid adjacent to the pitching plate such that the stabilizing foot grid is sandwiched between the drive-lane grid and the pitching plate, and such that when positioning the one or more foot pads upon the mat, a first foot pad is positioned in the stabilizing foot grid adjacent to the pitching plate and a second foot pad is positioned in the drive-lane grid.

In yet another aspect, the first foot pad is positioned in an orientation substantially parallel to a longitudinal axis of the pitching plate, while the second foot pad is position in an orientation that is substantially perpendicular to the first foot pad.

In yet another aspect, the plurality of grid zones includes a sweep grid adjacent to the drive-lane grid such that when positioning the one or more foot pads upon the mat, a third foot pad is positioned within the sweep grid adjacent to the second foot pad.

Further, when positioning one or more feet upon the one or more foot pads, a user's rear foot is positioned upon the first foot pad.

In yet another aspect, performing a pitching motion while moving feet between the one or more foot pads further comprises acts of raising a front stride foot; stretching the front stride foot forward until placing the front stride foot upon the second foot pad; and bringing the rear foot forward until the rear foot lands upon the third foot pad.

In another aspect, while stretching the front stride foot forward until placing the front stride foot upon the second foot pad, the user further performs an action of moving the user's gloved hand in a sweeping motion over a sweeping arrow formed on the mat that leads away from the pitching plate and forward through the one or more sweep grids.

In another aspect, the third foot pad is positioned within the drive-lane grid in an orientation that is substantially parallel to the first foot pad.

In yet another aspect, when positioning one or more feet upon the one or more foot pads, a user's rear foot is positioned upon the first foot pad while the user's front stride foot is positioned on the third foot pad.

In another aspect, performing a pitching motion while moving feet between the one or more foot pads further comprises acts of raising the front stride foot from the third foot pad; and stretching the stride foot forward until placing the front stride foot upon the second foot pad while moving the user's gloved hand in a sweeping motion over a sweeping arrow formed on the mat that leads away from the pitching plate and forward through the one or more sweep grids.

Finally, as can be appreciated by one in the art, the present invention also comprises a method for forming the invention described herein.

### BRIEF DESCRIPTION OF THE DRAWINGS

The objects, features and advantages of the present invention will be apparent from the following detailed descriptions of the various aspects of the invention in conjunction with reference to the following drawings, where:

FIG. 1 is a top, isometric view illustration of a sports training mat according to various embodiments of the present invention, depicting an aspect designed for both left hand and right hand pitchers;

FIG. 2 is a top-view illustration of the sports training mat as depicted in FIG. 2;

FIG. 3 is a top, isometric view illustration of a sports training mat according to various embodiments of the present invention, depicting an aspect designed for right hand pitchers;

FIG. 4 is a top-view illustration of the sports training mat as depicted in FIG. 3;

FIG. 5 is a top, isometric view illustration of a sports training mat according to various embodiments of the present invention, depicting an aspect designed for left hand pitchers;

FIG. 6 is a top-view illustration of the sports training mat as depicted in FIG.

FIG. 7 is a top-view illustration of the sports training mat, showing grid zones for placement of the foot pads;

FIG. 8 is a top-view illustration of the sports training mat, showing foot pad placement for use in a first method for pitching; and

FIG. 9 is a top-view illustration of the sports training mat, showing foot pad placement for use in a second method for pitching.

### DETAILED DESCRIPTION

The present invention relates to a sports training system and, more specifically, to a sports training mat and foot pads that can be used to assist pitchers in identifying foot placement and mechanics during a pitching motion. The following description is presented to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of embodiments. Thus, the present invention is not intended to be limited to the embodiments presented, but is to be accorded the widest scope consistent with the principles and novel features disclosed herein.

In the following detailed description, numerous specific details are set forth in order to provide a more thorough understanding of the present invention. However, it will be apparent to one skilled in the art that the present invention may be practiced without necessarily being limited to these specific details. In other instances, well-known structures and devices are shown in block diagram form, rather than in detail, in order to avoid obscuring the present invention.

The reader's attention is directed to all papers and documents which are filed concurrently with this specification and which are open to public inspection with this specification, and the contents of all such papers and documents are incorporated herein by reference. All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is only one example of a generic series of equivalent or similar features.

Furthermore, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of” or “act of” in the claims herein is not intended to invoke the provisions of 35 U.S.C. 112, Paragraph 6.

Please note, if used, the labels left, right, front, back, top, bottom, forward, reverse, clockwise and counter clockwise have been used for convenience purposes only and are not intended to imply any particular fixed direction.

Instead, they are used to reflect relative locations and/or directions between various portions of an object. Further, it is understood that when a first element is referred to as being “between,” “sandwiched,” or “sandwiched between,” two or more other elements, the first element can be directly between the two or more other elements or intervening elements may also be present between the two or more other elements. For example, if a first element is “between” or “sandwiched between” a second and third element, the first element can be directly between the second and third elements with no intervening elements or the first element can be adjacent to one or more additional elements with the first element and these additional elements all between the second and third elements.

### (1) Description

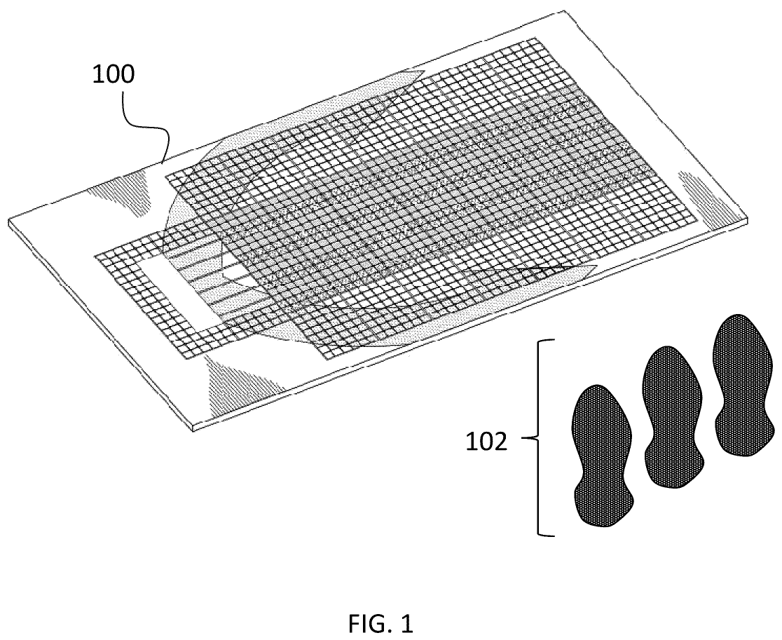

As shown in FIG. 1, the present disclosure provides a sports training system that can be used to assist pitchers in identifying foot placement and mechanics during a pitching motion. The sports training system includes a mat 100 that is made of any suitable material, non-limiting examples of which include felt, rubber, plastic, wood etc. The mat 100 can be used on any surface and, in some aspects, can be formed such that it is portable and rolls up for easy transportation and storage (e.g., formed of a flexible and rollable material such as rubber, fabric, felt, etc.). Also included in the sports training mat system are a plurality of foot pads 102, desirably three foot pads or any other number as desired. The foot pads 102 are formed to allow the foot pads 102 to be selectively repositioned and affixed with the mat 100 at any desired location. As a non-limiting example, the foot pads 102 include hook and loop fasteners that allow them to be affixed with the mat 100 at the desired location. As another example, the foot pads 102 include spikes or other features that allow them to press into and affix with the mat 100. In one aspect, the foot pads 102 are color-coded rubber foot markers that are specially designed to be placed according to each individual pitcher's and stride positioning on mat, including back stabilizing foot, front stride foot and drive foot landing pads. In one aspect, the mat 100 and foot pads 102 can be formed in a similar manner as the mat and any of the foot pads as depicted and described in U.S. Pat. No. 11,446,559 (the '559 patent), the entirety of which is incorporated by reference as though fully set forth herein.

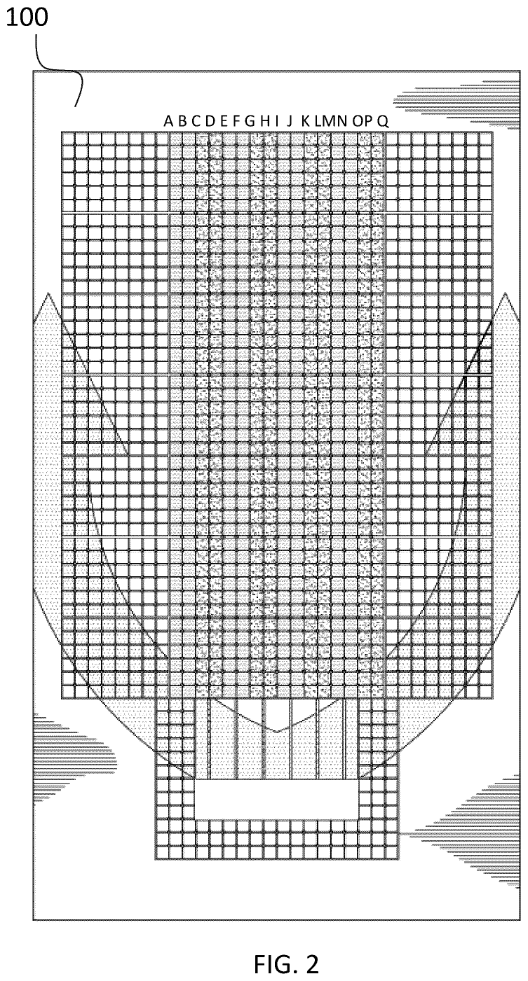

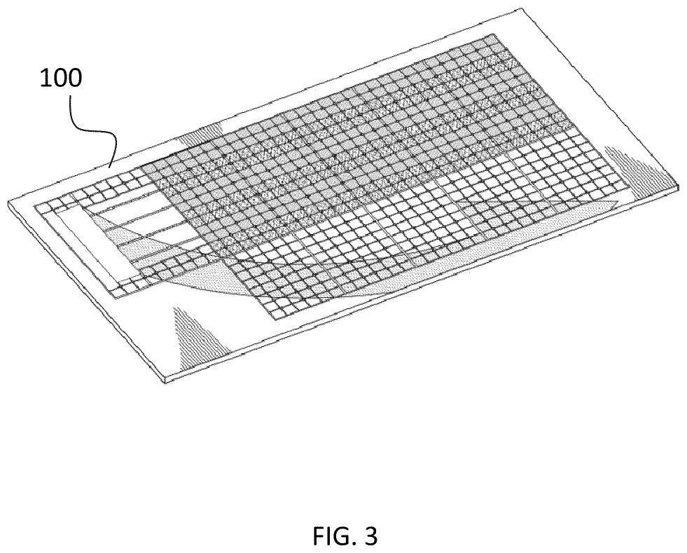

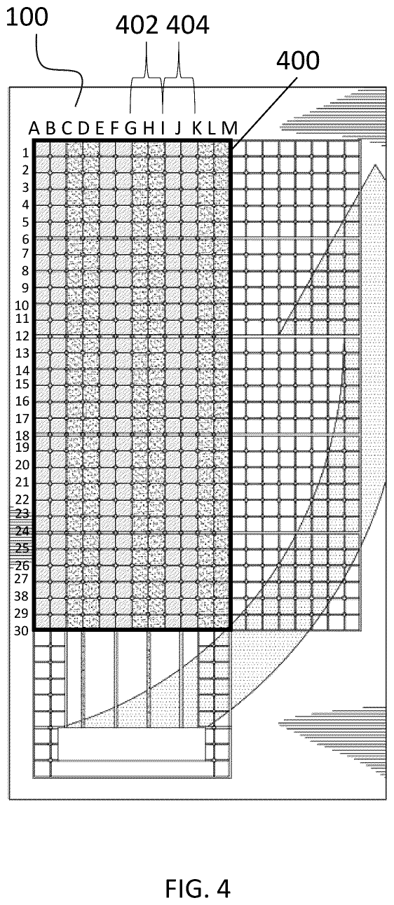

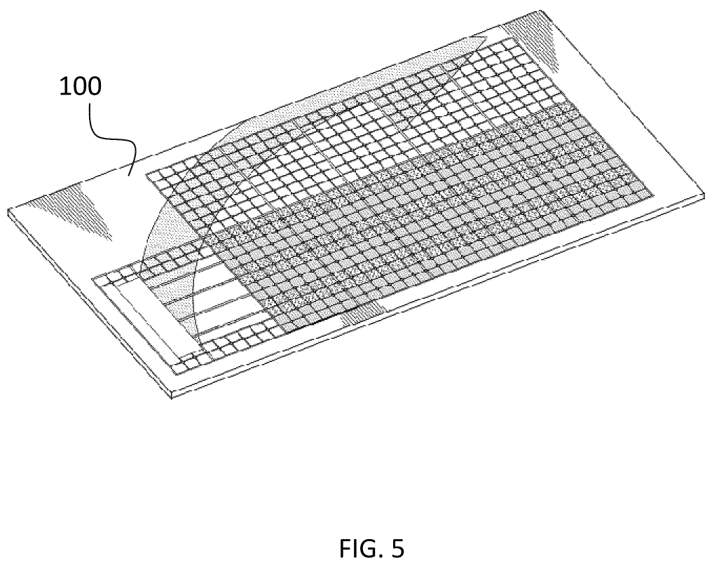

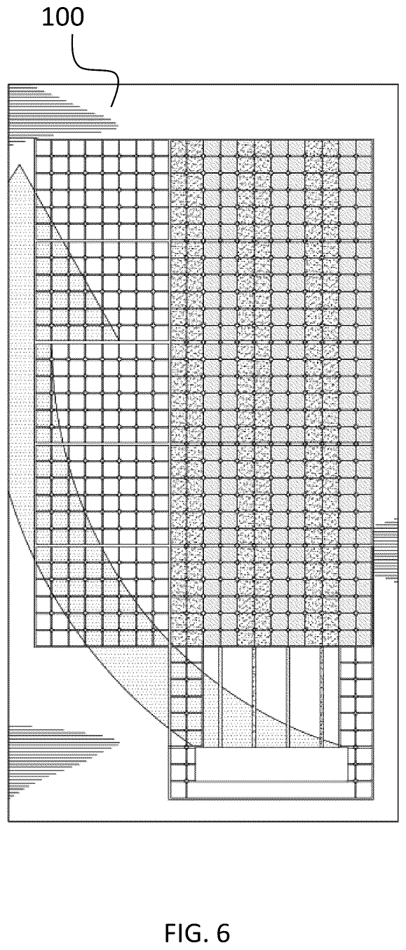

To be contrasted with the mat in the '559 patent, the mat 100 in the present disclosure is devised to assist a pitcher in performing a correct pitching motion by including additional zones with indicia (paint, markings, etc.) that assists with the foot placements when pitching. As can be appreciated by those skilled in the art, there are both right-handed and left-handed pitchers. Thus, in one aspect, the indicia required for a right-handed or left-handed pitcher may vary based on their orientation on the mat 100. For example, the mat 100 as depicted in FIGS. 1 and 2 includes indicia for both right-handed and left-handed pitchers, whereas FIGS. 3 and 4 depict the mat 100 as having indicia for a right-handed pitcher, while FIGS. 5 and 6 depict the mat 100 as having indicia for a left-handed pitcher. The indicia and use of the mat 100 are described in further detail below.

The mat 100 can be formed with any desired indicia (paint, coloration, markings, etc.) to allow a user to make and repeat foot or foot pad placement to ensure a consistent and repeated motion during pitching practice. In one aspect and as shown in FIG. 4, the mat 100 includes a plurality of grid zones formed thereon (e.g., painted, embossed, pressed, stitched, molded, etc.) to assist with foot placement. For example, the mat 100 includes a color-coded drive-lane grid 400 with numerical and alphabetical intersecting points distinguishing the “drive lanes” 402 that a baseball, softball or cricket pitcher would need to create neuro-muscle memory for their own personal bio-mechanical body sequenced movements. The drive lanes within the drive-lane grid are provided with indicia, such as colors, to distinguish a first set of drive lanes 402 from a second set of drive lanes 404, etc. As a non-limiting example, green can be used to distinguish the first set of drive lanes 402, while blue can be used to distinguish the second set of drive lanes 404. The colors can then alternate across the drive-lane grid 400, or used with any other color or indica scheme. The drive lanes serve as a series of colored parallel markings traveling away from a pitcher location to assist the pitcher in identifying linear foot placement or other placement as desired. Importantly, the drive lanes are clearly marked (e.g., with alternating colors) to help the pitcher while in the rapid forward pitching motion to maintain linear motion and foot placement.

It should be noted that although FIG. 4 includes numerical and/or alphabetical markings, such markings can be reversed, altered or otherwise provided as desired to allow a user to easily identify a particular grid location within the drive-lane grid 400 (or any other grid on the mat 100). Further, it should noted that such numerical and/or alphabetical markings can also be included in the other versions of mats as illustrated and described, including those as shown in FIGS. 2 and 6, or any combination thereof.

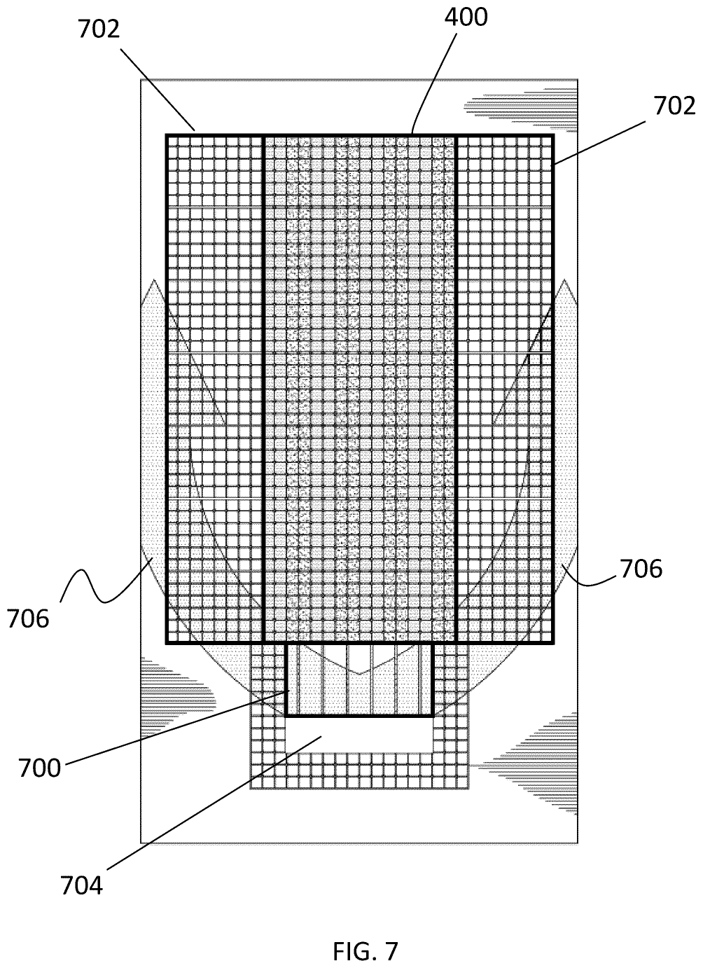

As noted above, the mat 100 can be formed with any grid pattern as desired to allow a user to mark and repeat foot or foot pad placement. Desirably and referring to FIG. 7, each mat 100 has a drive-lane grid 400, a stabilizing foot grid 700, and one or more sweep grids 702. The stabilizing foot grid 700 is framed on a peripheral edge by a pitching plate 704. The pitching plate 704 can be formed with white indicia or markings (e.g., white paint, or any other marking or color) on the mat 100 to reflect or otherwise mimic a traditional white pitching plate. In other aspects, a raised rubber plate is affixed with the mat 100 to serves as the pitching plate 704. In either event, the stabilizing foot grid 700 is sandwiched between the pitching plate 704 and drive-lane grid 400. In pitching, a user typically places their back stabilizing foot within the stabilizing foot grid 700 and, ideally, against the pitching plate 704. The grid or lined patterns within the stabilizing foot grid 700 allow a user to align their back stabilizing foot with a desired landing position for their front stride foot within the drive-lane grid 400.

Positioned along the sides of the drive-lane grid 400 are sweep grids 702. Further, extending from the stabilizing foot grid 700 and through the sweep grids 702 are sweeping arrows 706 that travel away from the pitching plate 704 and forward through the sweep grids 702. It should be noted that the aspect as depicted in FIG. 7 is designed to accommodate both right-handed and left-handed pitchers. As such, the design as depicted includes sweep grids 702 and sweeping arrows 706 along both sides of the drive-lane grid 400. However, in the right-hand aspect as shown in FIG. 4, and the left-hand aspect as shown in FIG. 6, the sweep grid 702 and corresponding sweeping arrow 706 are formed alongside the drive-lane-grid 400 in the direction that the pitcher is facing while using the mat 100.

The sweeping arrows 706 are included on the mat as indica (e.g., markings, paint, etc.) for visual direction to the pitcher. The visual aid provides the proper directional motion of the glove (glove should sweep out in the direction of the arrow 706 during the pitch motion) and back foot/drive foot as to move forward toward a landing position. The landing position when pitching is typically within the sweep grid 702. Thus, the sweep grid 702 includes a series of grid indicia to allow a user to place a foot pad thereon to indicate a desired landing position for the drive foot while pitching.

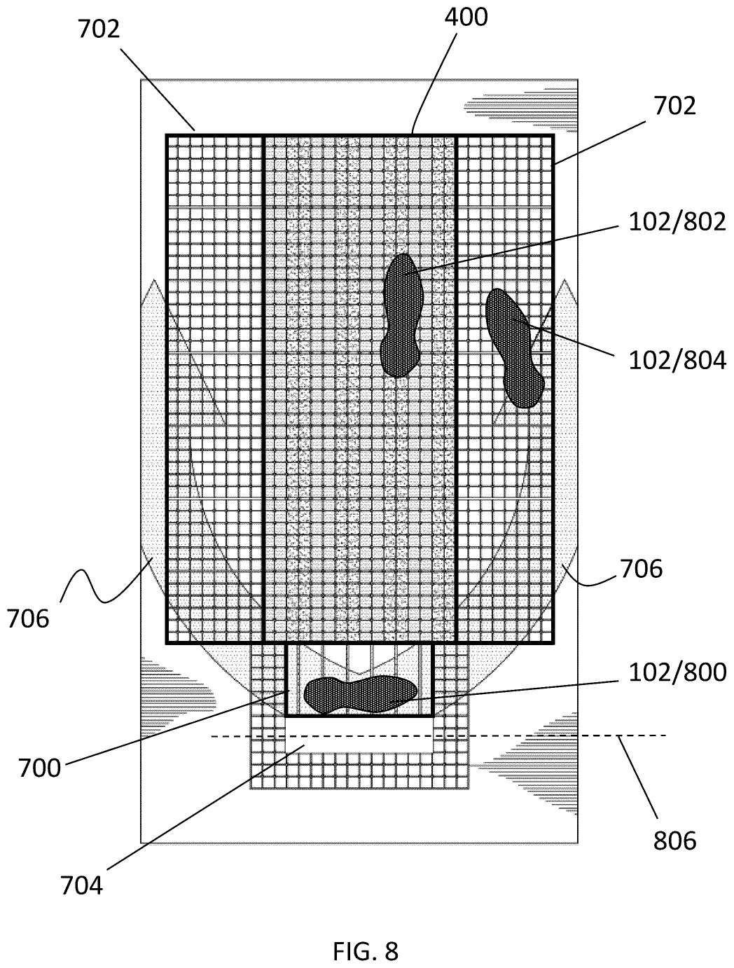

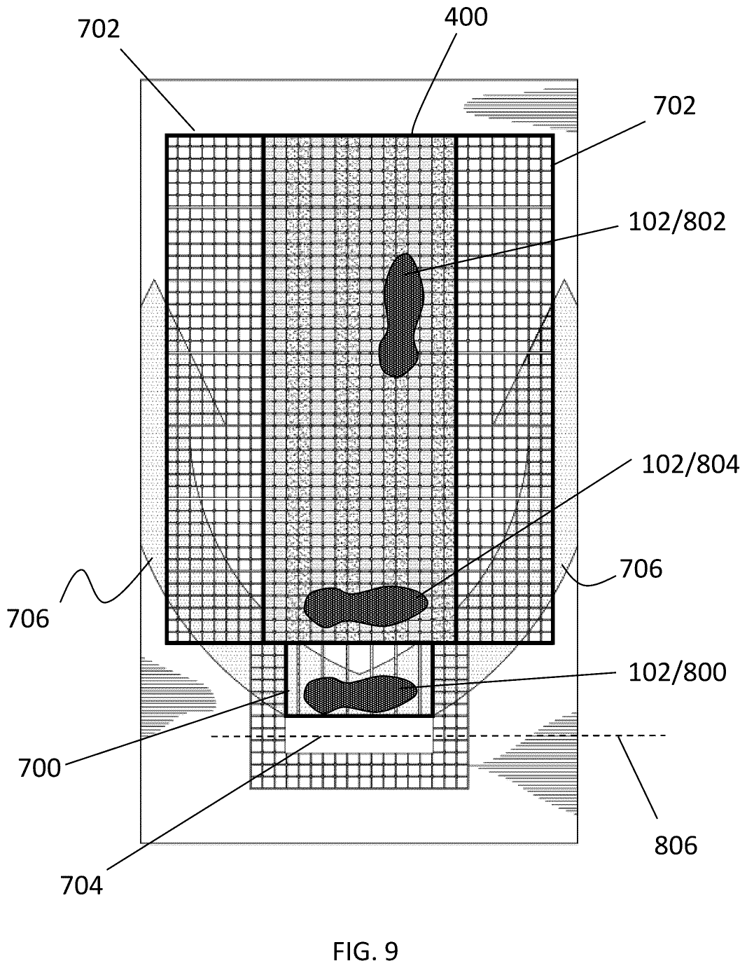

As can be appreciated by those skilled in the art, the foot pads 102 can be adhered or otherwise positioned on the mat 100 at any desired location to assist a user in pitching mechanics. Thus, there are a variety of methods by which the mat 100 can be used to train a pitcher and create neuro-muscle memory for their own personal bio-mechanical body sequenced movements. Two non-limiting examples of such a method are described in further detail below with respect to FIGS. 8 and 9.

In one aspect and as shown in FIG. 8, three foot pads 102 are used. For example, a first foot pad 800 is placed within the stabilizing foot grid 700 proximate to or against the pitching plate 704 in a direction that is parallel to the longitudinal axis 806 of the pitching plate 704. The first foot pad 800 is used to indicate a set position and placement of the back stabilizing foot. A second foot pad 802 is placed within the drive-lane grid 400 in an orientation generally perpendicular to that of the first foot pad 800 in a substantially forward pitching direction. The second foot pad 802 is used to indicate a stride point or landing point for the front stride foot when pitching. Finally, a third foot pad 804 is placed within the sweep grid 702 in a substantially forward orientation (i.e., in a pitching direction). As noted above, the third foot pad 802 is used to indicate a desired landing position for the back foot/drive foot when moving forward.

In the case of a right-handed pitcher, the rear foot (i.e., right foot) would start on the first foot pad 800 in a rocker step position. As the pitcher starts the pitching motion, the pitcher raises a lead or stride foot (i.e., left foot). The pitcher then stretches the lead/stride forward, with the lead/stride foot landing upon the second foot pad 802 (i.e., front or stride foot pad), while the momentum brings the rear foot forward to land upon the third foot pad 804 (drive or landing foot pad) adjacent to the second foot pad 802. While the left foot stretches forward towards the second foot pad 802, the pitcher's gloved hand (i.e., left hand) should sweep out in the direction of the arrow 706 during the pitch motion.

Another example of use is shown in FIG. 9. In this example, the first foot pad 800 is placed within the stabilizing foot grid 700 parallel to the longitudinal axis 806 of the pitching plate 704 while the third foot pad 804 is placed within the drive-lane grid 400 adjacent to the stabilizing foot grid 700 and in an orientation parallel to the first foot pad 800. Finally, the second foot pad 802 is placed within the drive-lane grid 400 in an orientation generally directed in the pitching direction (i.e., perpendicular to the first foot pad 800). In use, the pitcher would place his/her right foot on the first foot pad 800 and left foot on the third foot pad 804 in a starting position. The pitcher would then raise a front leg and move the front leg in a sweeping stretch motion out over the drive-lane grid 400 and toward the stride foot pad (i.e., second foot pad 802) such that the right foot rests upon the first foot pad 800 (and optionally rises therefrom) while bringing the pitching arm forward to release the pitch. In the case of a left-handed pitcher, the left and right feet and gloved hand in both examples above would be switched to reflect a left-handed pitch.

Finally, while this invention has been described in terms of several embodiments, one of ordinary skill in the art will readily recognize that the invention may have other applications in other environments. It should be noted that many embodiments and implementations are possible. Further, the following claims are in no way intended to limit the scope of the present invention to the specific embodiments described above. In addition, any recitation of “means for” is intended to evoke a means-plus-function reading of an element and a claim, whereas, any elements that do not specifically use the recitation “means for”, are not intended to be read as means-plus-function elements, even if the claim otherwise includes the word “means”. Further, while particular method steps have been recited in a particular order, the method steps may occur in any desired order and fall within the scope of the present invention.

What is claimed is:

1. A sports training system, comprising:
a mat having a pitching plate and a plurality of grid zones formed thereon; and
a plurality of foot pads for placement in one or more of the plurality of grid zones.

2. The sports training system as set forth in claim 1, wherein the plurality of grid zones includes a drive-lane grid, the drive-lane grid having a series of parallel markings traveling away from the pitching plate.

3. The sports training system as set forth in claim 2, wherein the plurality of grid zones includes a stabilizing foot grid adjacent to the pitching plate such that the stabilizing foot grid is sandwiched between the drive-lane grid and the pitching plate.

4. The sports training system as set forth in claim 3, wherein the plurality of grid zones includes one or more sweep grids adjacent to the drive-lane grid.

5. The sports training system as set forth in claim 4, further comprising one or more sweeping arrows formed on the mat, the one or more sweeping arrows leading away from the pitching plate and forward through the one or more sweep grids.

6. A method for using a sports training system during pitching lessons, comprising acts of:
positioning one or more foot pads upon a mat, the mat having a pitching plate and a plurality of grid zones;
positioning one or more feet upon the one or more foot pads; and
performing a pitching motion while moving feet between the one or more foot pads.

7. The method as set forth in claim 6, wherein the plurality of grid zones includes at least a drive-lane grid having a series of parallel markings traveling away from the pitching plate and a stabilizing foot grid adjacent to the pitching plate such that the stabilizing foot grid is sandwiched between the drive-lane grid and the pitching plate, and such that when positioning the one or more foot pads upon the mat, a first foot pad is positioned in the stabilizing foot grid adjacent to the pitching plate and a second foot pad is positioned in the drive-lane grid.

8. The method as set forth in claim 7, wherein the first foot pad is positioned in an orientation substantially parallel to a longitudinal axis of the pitching plate, while the second foot pad is position in an orientation that is substantially perpendicular to the first foot pad.

9. The method as set forth in claim 8, wherein the plurality of grid zones includes a sweep grid adjacent to the drive-lane grid such that when positioning the one or more foot pads upon the mat, a third foot pad is positioned within the sweep grid adjacent to the second foot pad.

10. The method as set forth in claim 9, wherein when positioning one or more feet upon the one or more foot pads, a user's rear foot is positioned upon the first foot pad.

11. The method as set forth in claim 10, wherein performing a pitching motion while moving feet between the one or more foot pads further comprises acts of:
raising a front stride foot;
stretching the front stride foot forward until placing the front stride foot upon the second foot pad; and
bringing the rear foot forward until the rear foot lands upon the third foot pad.

12. The method as set forth in claim 11, wherein while stretching the front stride foot forward until placing the front stride foot upon the second foot pad, moving the user's gloved hand in a sweeping motion over a sweeping arrow formed on the mat that leads away from the pitching plate and forward through the one or more sweep grids.

13. The method as set forth in claim 8, wherein the third foot pad is positioned within the drive-lane grid in an orientation that is substantially parallel to the first foot pad.

14. The method as set forth in claim 13, wherein when positioning one or more feet upon the one or more foot pads, a user's rear foot is positioned upon the first foot pad while the user's front stride foot is positioned on the third foot pad.

15. The method as set forth in claim 14, wherein performing a pitching motion while moving feet between the one or more foot pads further comprises acts of:
raising the front stride foot from the third foot pad; and
stretching the stride foot forward until placing the front stride foot upon the second foot pad while moving the user's gloved hand in a sweeping motion over a sweeping arrow formed on the mat that leads away from the pitching plate and forward through the one or more sweep grids.
